(19) United States
(12) Patent Application Publication
(10) Pub. No.: US 2021/0046140 A1
(43) Pub. Date: Feb. 18, 2021

(54) PHARMACEUTICAL COMPOSITION AND DIETARY SUPPLEMENT FOR PREVENTING OR TREATING OVERACTIVE BLADDER CAUSED BY SECONDARY BLADDER DEGENERATION DUE TO BLADDER OUTLET OBSTRUCTION CAUSED BY BENIGN PROSTATIC HYPERPLASIA

(71) Applicants: KEMIMEDI CO. LTD, Gyeongsangbuk-do (KR); THE CATHOLIC OF KOREA INDUSTRY-ACADEMIC COOPERATION FOUNDATION, Seoul (KR)

(72) Inventors: Sung Yeoun HWANG, Incheon (KR); Sae Woong KIM, Seoul (KR)

(21) Appl. No.: 16/087,375

(22) PCT Filed: Mar. 24, 2017
(86) PCT No.: PCT/KR2017/003228

§ 371 Date: Sep. 21, 2018

(30) Foreign Application Priority Data
Mar. 24, 2016 (KR) 10-2016-0035262
Mar. 24, 2017 (KR) 10-2017-0037929

(51) Int. Cl. A61K 36/815; A23L 33/105; A23L 33/00; A61K 36/40; A61K 36/232; A61K 35/12; A61K 36/258; A61K 36/54; A61K 31/517; A61K 31/18; A61P 13/10

(52) U.S. Cl. CPC A61K 36/815; A23L 33/105; A23L 33/40; A61K 36/40; A61K 36/232; A23V 2002/00; A61K 36/258; A61K 36/54; A61K 31/517; A61K 31/18; A61P 13/10; A61K 35/12

(57) ABSTRACT

A method for preventing or treating overactive bladder includes administering a composition to a subject in need thereof. The composition includes extracts having a Corni Fructus extract, an Angelica gigantis Radix extract, a Lycii Fructus extract, a Cervi parvum cornu extract, a Ginseng Radix Rubra extract, and a Cassiae cortkex extract. The overactive bladder may be a complication of benign prostatic hyperplasia, and the composition may further include an alpha-1A adrenoceptor antagonist. The overactive bladder may be caused by secondary bladder degeneration occurring due to bladder outlet obstruction. The composition may be included in a pharmaceutical composition or a health functional food. The composition also exhibits an excellent effect on overactive bladder that is not relieved even when bladder outlet obstruction disappears by benign prostatic hyperplasia surgical treatment or administration of an alpha-blocker or the like.

### TECHNICAL FIELD

The present invention relates to a pharmaceutical composition and health functional food for preventing or treating overactive bladder caused by secondary bladder degeneration due to bladder outlet obstruction induced by benign prostatic hyperplasia.

### BACKGROUND ART

A representative example of dysuria-related diseases is benign prostatic hyperplasia. Benign prostatic hyperplasia is seen in 40% to 70% of the elderly in their 60s and the main symptom is hyperplasia and hypertrophy of epithelial and interstitial tissues of the prostate. Factors regulating the proliferation and death of prostate cells include endocrine factors, various growth factors, interactions between interstitial cells and epithelial cells, neuroendocrine cells and neurotransmitters, substances that regulate cell cycle and death, inflammation, and the like.

When the size of the prostate gland increases, it blocks the urethra and causes bladder outlet obstruction, which makes urination difficult, and thus urination malfunctions, and secondly, overactive bladder symptoms, in which urinary continence does not function properly, such as frequent urination, urinary urgency, and the like may occur by detrusor overactivity. In addition, if bladder outlet obstruction persists for a long time, the contraction force of the detrusor muscle becomes inappropriate, resulting in an increase in the amount of residual urine, urinary retention, and the like, and consequently, complications such as urinary tract infection, urinary calculus, renal failure, and the like occur.

Overactive bladder (OAB) is a syndrome of urinary urgency as a main symptom, which is the feeling of an uncontrollable and sudden desire to urinate, and may or may not be accompanied by urinary incontinence. According to a report, the prevalence of overactive bladder in patients with benign prostatic hyperplasia was 45%, 55% of total patients with lower urinary tract symptoms purely showed only bladder outlet obstruction, and since there is a strong correlation between bladder outlet obstruction and overactive bladder, as the bladder outlet obstruction becomes severe, the percentage of overactive bladder increases. In addition, patients with benign prostatic hyperplasia have urination symptoms due to bladder outlet obstruction and, in half or more of the cases, storage symptoms (particularly overactive bladder symptoms) due to exacerbation of bladder outlet obstruction, and these symptoms are increasingly exacerbated due to an increase in detrusor overactivity as age increases, and it is known that one third or more of the elderly population has overactive bladder when they are 60 years old or older regardless of gender. Most of these patients control the frequency of urination by reducing water intake, but this may also reduce external activity.

Lower urinary tract symptoms (LUTs) refer to urinary storage and voiding disturbances, and are caused by dysuria such as benign prostatic hyperplasia, overactive bladder, neurogenic disorders, and the like. Patients with benign prostatic hyperplasia have two types of problems depending on the progression of a disease because the bladder outlet is blocked and thus urination does not properly function. First, an increase in internal pressure of the bladder initially induces overactivity of the bladder muscles, which makes the bladder sensitive, resulting in the occurrence of storage symptoms such as frequent urination, nocturia, urinary urgency, urge incontinence, dysuria, and the like. Second, when bladder outlet obstruction persists for a long time, the bladder muscles become thick so that the bladder is unable to contract, making it impossible to completely discharge urine in the bladder, resulting in the occurrence of residual urine or obstructive urination symptoms such as weak urinary stream, urination delay, urinary retention, and the like. If this condition persists, the bladder will eventually become unable to function, and thus when normal urination hardly functions, urinary retention may be caused.

The bladder contracts by activating the muscarinic receptor of the bladder body mainly by the stimulation of parasympathetic nerves, and sympathetic nerves relax the bladder through the β-receptor in the bladder body and the bladder is contracted through the purine receptor rather than the parasympathetic and sympathetic nerves. In addition, in the bladder neck and the prostate urethra, the sympathetic nerves cause contraction through the al receptor, and the external urethral sphincter located in the membranous urethra voluntarily regulates contraction and relaxation by somatic nerves.

Currently, alpha-blockers are drugs most widely used for the treatment of benign prostatic hyperplasia. Among these alpha-blockers, alpha-blockers which selectively act on the alpha-1A adrenoceptor (tamsulosin, doxazosin, alfuzosin, and the like) relax the prostate smooth muscle and reduce bladder neck obstruction, leading to relief of obstructive voiding symptoms (weak urinary stream, urination delay, incomplete urination, urinary pause, terminal dribbling, urinary retention, and the like). However, even when bladder outlet obstruction is relieved by such an alpha-blocker, there are cases in which storage symptoms (frequent urination, nocturia, urinary urgency, urgency, urinary incontinence, dysuria, and the like) persist. Thus, to relieve these irritative storage symptoms, alpha-blockers are administered in combination with drugs such as anticholinergic agents (e.g., parasympatholytics such as tolterodine, solifenacin, and the like), antipurinergic agents (propiverine HCl), and the like.

Anticholinergic agents regulate bladder overactivity during the filling phase of the micturition cycle, thereby allowing urinary continence and increasing bladder capacity. As anticholinergic agents, fesoterodine, solifenacin, tolterodine, trospium, oxybutynin, propiverine, and the like are currently used commonly in clinical trials. An important consideration to be taken into in the use of anticholinergic agents for the treatment of overactive bladder, together with therapeutic effects thereof is side effects due to the anticholinergic agents.

Examples of adverse reactions, which may occur when anticholinergic agents are used, include gastrointestinal pain, gastritis, nausea, vomiting, blurred vision, urinary retention, voiding difficulty, dysuria, urinary tract infection, fatigue, somnolence, sedation, insomnia, confusion, cognitive impairment, depression, headaches, palpitation, tachycardia, hypertension, orthostatic disturbance, falls, and the like. Anticholinergic agents are contraindicated, especially for severe arrhythmia, constrictive glaucoma, obstructive diseases of the digestive system, myasthenia gravis, and the like. For overactive bladder symptoms not improved even after benign prostatic hyperplasia surgical treatment, which is the case of bladder overactivity remaining even after the treatment of bladder outlet obstruction due to benign prostatic hyperplasia, an additional drug treatment is needed. Due to side effects of these existing anticholinergic agents, it is difficult for elderly patients to take these drugs for a long time, and thus, when a therapeutic agent or a health function food, which can replace anticholinergic agents, is derived from a natural substance, is safe, and has scientifically verified efficacy, thus enabling long-term use thereof, is developed to relieve overactive bladder symptoms that persist even after benign prostatic hyperplasia treatment, it will be a great help to many patients with benign prostatic hyperplasia accompanied by overactive bladder symptoms.

### DISCLOSURE

### Technical Problem

An object of the present invention is to provide a pharmaceutical composition for preventing or treating overactive bladder caused by secondary bladder degeneration due to bladder outlet obstruction induced by benign prostatic hyperplasia.

Another object of the present invention is to provide a health functional food for preventing or alleviating overactive bladder caused by secondary bladder degeneration due to bladder outlet obstruction induced by benign prostatic hyperplasia.

### Technical Solution

According to an aspect of the present invention, there is provided a pharmaceutical composition for preventing or treating overactive bladder, which is a complication of benign prostatic hyperplasia, the pharmaceutical composition including Corni Fructus, Angelica gigantis Radix, Lycii Fructus, Cervi parvum cornu, Ginseng Radix Rubra, and Cassiae cortkex extracts, and an alpha-1A adrenoceptor antagonist.

The overactive bladder is caused by secondary bladder degeneration occurring due to bladder outlet obstruction.

The overactive bladder is not relieved even after bladder outlet obstruction disappears.

The extracts include 20 parts by weight to 30 parts by weight of Corni Fructus, 20 parts by weight to 30 parts by weight of Angelica gigantis Radix, 20 parts by weight to 30 parts by weight of Lycii Fructus, 5 parts by weight to 15 parts by weight of Cervi parvum cornu, 5 parts by weight to 15 parts by weight of Ginseng Radix Rubra, and 2 parts by weight to 8 parts by weight of Cassiae cortkex.

According to another aspect of the present invention, there is provided a health functional food for preventing or alleviating overactive bladder, which is a complication of benign prostatic hyperplasia, the health functional food being ingestible when an alpha-1A adrenoceptor antagonist is administered and including Corni Fructus. Angelica gigantis Radix, Lysii Fructus, Cervi parvum cornu. Ginseng Radix Rubra, and Cassiae cortkex extracts.

The overactive bladder is caused by secondary bladder degeneration occurring due to bladder outlet obstruction.

According to another aspect of the present invention, there is provided a pharmaceutical composition for preventing or treating overactive bladder caused by secondary bladder degeneration occurring due to bladder outlet obstruction, the pharmaceutical composition including Corni Fructus, Angelica gigantis Radix, Lycii Fructus, Cervi parvum cornu, Ginseng Radix Rubra, and Cassiae cortkex extracts.

The overactive bladder is not relieved even after bladder outlet obstruction disappears.

The extracts include 20 parts by weight to 30 parts by weight of Corni Fructus, 20 parts by weight to 30 parts by weight of Angelica gigantis Radix, 20 parts by weight to 30 parts by weight of Lycii Fructus, 5 parts by weight to 15 parts by weight of Cervi parvum cornu, 5 parts by weight to 15 parts by weight of Ginseng Radix Rubra, and 2 parts by weight to 8 parts by weight of Cassiae cortkex.

### Advantageous Effects

A pharmaceutical composition of the present invention exhibits an excellent effect on overactive bladder caused by secondary bladder degeneration due to bladder outlet obstruction induced by benign prostatic hyperplasia. In particular, the pharmaceutical composition can also exhibit an excellent effect on overactive bladder that is not relieved even when bladder outlet obstruction disappears by benign prostatic hyperplasia surgical treatment or administering an alpha-blocker or the like.

A health functional food of the present invention exhibits excellent functionality in overactive bladder symptoms caused by secondary bladder degeneration due to bladder outlet obstruction induced by benign prostatic hyperplasia. In particular, the health functional food can also exhibit excellent functionality in overactive bladder that is not relieved even when bladder outlet obstruction disappears by benign prostatic hyperplasia surgical treatment or administering an alpha-blocker or the like.

### DESCRIPTION OF DRAWINGS

FIG. 1 illustrates urodynamic examination results after treatment with a composition according to an embodiment of the present invention.

FIG. 2 illustrates histologic examination results after treatment with a composition according to an embodiment of the present invention.

FIG. 3 illustrates measurement results of the concentrations of inflammatory cytokines after treatment with a composition according to an embodiment of the present invention.

FIG. 4 illustrates measurement results of the expression of proteins associated with contraction of the bladder smooth muscle after treatment with a composition according to an embodiment of the present invention.

FIG. 5 illustrates mRNA expression measurement results of muscarinic receptors associated with bladder smooth muscle contraction after treatment with a composition according to an embodiment of the present invention.

FIG. 6 illustrates measurement results of mRNA expression associated with bladder smooth muscle contraction after treatment with a composition according to an embodiment of the present invention.

### MODE

The present invention includes two or more selected from the group consisting of a Corni Fructus extract, an Angelica gigantis Radix extract, a Lycii Fructus extract, a Cervi parvum cornu extract, a Ginseng Radix Rubra extract, and a Cassiae cortkex extract and an alpha-1A adrenoceptor antagonist, and thus provides an excellent effect on overactive bladder caused by secondary bladder degeneration due to bladder outlet obstruction induced by benign prostatic hyperplasia. In particular, the present invention relates to a pharmaceutical composition and health functional food for preventing or treating overactive bladder, which is a complication of benign prostatic hyperplasia, the pharmaceutical composition and health functional food being capable of exhibiting an excellent effect on overactive bladder that is not relieved even when bladder outlet obstruction disappears by benign prostatic hyperplasia surgical treatment or administration of an alpha-blocker or the like.

Hereinafter, the present invention will be described in detail.

In the present specification, benign prostatic hyperplasia is closely related to aging and male hormones, and about 40% to about 70% of men aged 60 years or older have lower urinary tract symptoms due to benign prostatic hyperplasia and this seriously affects their quality of life. This disease is mainly seen in men aged 50 years or older, and patients with this disease have lower urinary tract symptoms including: bladder irritative storage symptoms such as urinary frequency that occurs 8 times or more a day, nocturia, urinary urgency which is the feeling of a sudden desire to urinate and is uncontrollable when this feeling occurs, and the like; obstructive voiding symptoms exhibiting bladder voiding disorders such as urinary hesitancy (a condition where a patient has to wait for the urinary stream for a certain amount of time), disconnected urine (a condition where the urinary stream stops), a condition where force is needed during urination, and the like.

In the present specification, overactive bladder is a secondary change caused by benign prostatic hyperplasia, and when the size of the prostate gland increases, the urethra is blocked, and thus bladder outlet obstruction, in which urinary stream is unable to be discharged, is caused. At this time, pressure in the bladder increases and it causes overactivity of the bladder muscle, leading to a sensitive bladder, and as a result, storage symptoms. i.e., overactive bladder symptoms such as urinary frequency, nocturia, urinary urgency, urge incontinence, and the like. In the present specification, overactive bladder refers to overactive bladder caused by secondary bladder degeneration occurring due to bladder outlet obstruction.

For the overactive bladder, which is a complication of benign prostatic hyperplasia, general methods of treating benign prostatic hyperplasia, such as administration of an alpha-1A adrenoceptor antagonist (an alpha-blocker, a drug that specifically blocks the action of the α-receptor of an adrenergic agonist), an adrenergic inhibitor, or the like, benign prostatic hyperplasia surgical treatment, and the like, have been used, but there are cases in which, even when bladder outlet obstruction is relieved, overactive bladder still persists.

Meanwhile, when an anticholinergic drug, which is an overactive bladder therapeutic agent, is administered in combination to inhibit this condition, sides effects such as dry mouth, constipation, blurred vision, drowsiness, cognitive disorders, gastrointestinal disorders, and the like occur, and thus most patients stop taking the medication or are unable to take the drug from the beginning, and therefore, there is a need for a novel therapeutic agent.

The inventors of the present invention discovered that, when an alpha-1A adrenoceptor antagonist and Corni Fructus, Angelica gigantis Radix, Lycii Fructus, Cervi parvum cornu, Ginseng Radix Rubra, and Cassiae cortkex extracts are co-administered, overactive bladder, which is a complication of benign prostatic hyperplasia, particularly overactive bladder that is not relieved even when bladder outlet obstruction disappears by benign prostatic hyperplasia surgical treatment or administration of an alpha-blocker or the like, could be effectively suppressed without the above-described side effects, thus completing the present invention based on these findings.

The pharmaceutical composition for preventing or treating overactive bladder caused by secondary bladder degeneration due to bladder outlet obstruction induced by benign prostatic hyperplasia of the present invention includes Corni Fructus, Angelica gigantis Radix, Lycii Fructus, Cervi parvum cornu, Ginseng Radix Rubra, and Cassiae cortex extracts, and an alpha-1A adrenoceptor antagonist.

A ratio of amounts of the extracts is not particularly limited, and the extracts may include, for example, 20 parts by weight to 30 parts by weight of Corni Fructus, 20 parts by weight to 30 parts by weight of Angelica gigantis Radix, 20 parts by weight to 30 parts by weight of Lycii Fructus, 5 parts by weight to 15 parts by weight of Cervi parvum cornu, 5 parts by weight to 15 parts by weight of Ginseng Radix Rubra, and 2 parts by weight to 8 parts by weight of Cassiae cortkex.

Each extract may be obtained by drying and extracting each medicinal ingredient, if needed, followed by concentration and lyophilization.

The extracting process may be performed by hot water extraction using purified water, or may be performed using a C₁-C₄ lower alcohol solvent or one or more hydrocarbon solvents selected from methyl acetate, ethyl acetate, benzene, n-hexane, diethyl ether, and dichloromethane, and the extracting process may be performed at 40° C. to 100° C. for 1 hour to 6 hours, preferably using 10% to 50% ethanol at 60° C. to 100° C. for 3 hours to 6 hours.

An alpha-1A adrenoceptor antagonist lowers the pressure and tension of the prostate urethra. Examples of suitable and commercially available alpha-blockers include terazosin, doxazosin, tamsulosin, alfuzosin, and the like.

The pharmaceutical composition of the present invention may be formulated in the form of oral preparations such as powder, granules, tablets, capsules, suspensions, emulsions, syrups, aerosol, and the like, preparations for external application, suppositories, and sterile injection solutions, according to general methods.

Examples of carriers, excipients, and diluents that may be included in the composition including the extracts of the present invention may include lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, and mineral oil. The pharmaceutical composition is formulated using a diluent or excipient such as a filler, an extender, a binder, a wetting agent, a disintegrant, a surfactant, or the like that is commonly used.

Solid preparations for oral administration include tablets, pills, powder, granules, capsules, and the like, and such solid preparations are formulated by mixing the compound with at least one excipient, e.g., starch, calcium carbonate, sucrose, lactose, gelatin, and the like. In addition to simple excipients, lubricants such as magnesium stearate and talc are also used. Examples of liquid preparations for oral administration include suspensions, liquids for internal use, emulsions, syrups, and the like, and these liquid preparations may include, in addition to commonly used simple diluents, such as water and liquid paraffin, various types of excipients, for example, a wetting agent, a sweetener, a flavoring agent, a preservative, and the like.

Preparations for parenteral administration include an aqueous sterile solution, a non-aqueous solvent, a suspension, an emulsion, a freeze-dried preparation, and a suppository. Examples of the non-aqueous solvent and the suspension include propylene glycol, polyethylene glycol, a vegetable oil such as olive oil, and an injectable ester such as ethyl oleate. Examples of suppository bases include WITEPSOL, MACROGOL, TWEEN 61, cacao butter, laurin, glycerogelatin, and the like.

A dose of the pharmaceutical composition of the present invention may vary depending on the age, gender, and body weights of patients, but the pharmaceutical composition may be administered once or multiple times a day in an amount of 0.1 mg/kg to 100 mg/kg, preferably 1 mg/kg to 10 mg/kg. In addition, the dose may be increased or decreased depending on administration route, the severity of diseases, gender, body weight, age, and the like. Therefore, the dose is not intended to limit the scope of the present invention in any way.

The present invention also provides a health functional food for preventing or alleviating overactive bladder caused by secondary bladder degeneration due to bladder outlet obstruction induced by benign prostatic hyperplasia, the health functional food being ingestible along with an alpha-1A adrenoceptor antagonist and including two or more selected from the group consisting of a Corni Fructus extract, an Angelico gigantis Radix extract, a Lycii Fructus extract, a Cervi parvum cornu extract, a Ginseng Radix Rubra extract, and a Cassiae cortkex extract.

Each extract may be obtained using the above-described method, and the extracts may be included in the above-described amount ratio, but the present invention is not limited thereto.

The health functional food of the present invention is ingestible along with an alpha-1A adrenoceptor antagonist, and for example, may be taken simultaneously when the alpha-1A adrenoceptor antagonist is administered, or may be taken during the period of administration of the alpha-1A adrenoceptor antagonist.

In a case in which the health functional food is taken during the period of administration of the alpha-1A adrenoceptor antagonist, when periods, in which an effect of the alpha-1A adrenoceptor antagonist and the functionality of the health functional food can be expressed, overlap with each other in consideration of the half-life in the body of the alpha-1A adrenoceptor antagonist and the half-life in the body of an effective ingredient of the health functional food of the present invention, administration time, administration period, and the like are not limited.

If needed, the health functional food of the present invention may further include an alpha-1A adrenoceptor antagonist, and the above-described commercially available alpha-blockers may be used as the alpha-A adrenoceptor antagonist, but the present invention is not limited thereto.

Examples of the health functional food of the present invention include beverages including the extracts (including alcoholic beverages), fruits and processed foods thereof (e.g., canned fruits, bottled foods, jam, marmalade, and the like), fish, meat and processed foods thereof (e.g., ham, sausage, corn beef, and the like), bread and noodles (e.g., Japanese-style noodles, buckwheat noodles, ramen, spaghetti, macaroni, and the like), fruit juices, various drinks, cookies, taffy, dairy products (e.g., butter, cheese, and the like), edible vegetable oils, margarine, vegetable proteins, retort foods, frozen foods, various seasonings (e.g., soybean paste, soy sauce, other sauces, and the like), and the like.

In addition, the health functional food of the present invention may be formulated into tablets, pills, powder, granules, capsules, liquid preparations, and the like. These may be formulated by further including one or more of carriers, diluents, excipients, and additives.

As an additive that may be further included in the present invention, one or more ingredients selected from the group consisting of natural carbohydrates, flavorings, nutrients, vitamins, minerals (electrolytes), flavors (synthetic flavors, natural flavors, and the like), colorants, enhancers (cheese, chocolate, and the like), pectic acid and salts thereof, alginic acid and salts thereof, organic acids, a protective colloid thickener, a pH adjuster, a stabilizer, a preservative, an antioxidant, glycerin, alcohols, a carbonating agent, and flesh may be used.

Examples of the aforementioned natural carbohydrates include general saccharides such as monosaccharides, e.g., glucose, fructose, and the like; disaccharides, e.g., maltose, sucrose, and the like; and polysaccharides, e.g., dextrin, cyclodextrin, and the like, and sugar alcohols such as xylitol, sorbitol, erthritol, and the like. As the flavors, natural flavors (thaumatin and stevia extracts (e.g., Rebaudioside A, glycyrrhizin, and the like) and synthetic flavors (saccharin, aspartame, and the like) may be preferably used.

In addition to the above-described ingredients, the health functional food of the present invention may include various nutritional supplements, vitamins. minerals (electrolytes), flavors such as synthetic flavors, natural flavors, and the like, colorants and enhancers (cheese, chocolates, and the like), pectic acid and salts thereof, alginic acid and salts thereof, organic acids, a protective colloid thickener, a pH adjuster, a stabilizer, a preservative, glycerin, alcohols, a carbonating agent used in carbonated beverages, and the like. In addition, the health functional food of the present invention may include flesh for the preparation of natural fruit juices and vegetable beverages. These ingredients may be used alone or a combination thereof may be used.

Specific examples of the carriers, the excipients, the diluents, and the additives may include, but are not limited to, one or more selected from the group consisting of lactose, dextrose, sucrose, sorbitol, mannitol, erythritol, starch, acacia rubber, alginate, gelatin, calcium phosphate, calcium silicate, microcrystalline cellulose, polyvinylpyrrolidone, cellulose, methyl cellulose, water, sugar syrup, methyl hydroxybenzoate, propyl hydroxybenzoate, talc, magnesium stearate, and mineral oil.

A content of the health functional food of the present invention as a functional ingredient in the above-described preparations may be appropriately adjusted depending on the form and purpose of use, the condition of a user, the type of symptoms, and the severity thereof, and may range from 0.001 wt % to 99.9 wt %, preferably 0.01 wt % to 50 wt % on a solid weight basis, but the present invention is not limited thereto.

The present invention also provides a pharmaceutical composition for preventing or treating overactive bladder caused by secondary bladder degeneration occurring due to bladder outlet obstruction, the pharmaceutical composition including Corni Fructus, Angelica gigantis Radix, Lycii Fructus, Cervi parvum cornu, Ginseng Radix Rubra, and Cassiae cortkex extracts.

As described above, an alpha-blocker is used to reduce the pressure and tension of the prostate urethra and relieve bladder outlet obstruction, and the Corni Fructus, Angelica gigantis Radix, Lycii Fructus, Cervi parvum cornu, Ginseng Radix Rubra, and Cassiae cortkex extracts may exhibit an effect on overactive bladder that is not relieved even after bladder outlet obstruction disappears, without an alpha-blocker.

The Corni Fructus. Angelica gigantis Radix, Lycii Fructus, Cervi parvum cornu, Ginseng Radix Rubra, and Cassiae cortkex extracts may also exhibit an effect on overactive bladder that is not relieved even after bladder outlet obstruction disappears.

The extracts may be included in the amount ratio within the above-described range.

The present invention also provides a health functional food for preventing or alleviating overactive bladder caused by secondary bladder degeneration occurring due to bladder outlet obstruction, the health functional food including Corni Fructus, Angelica gigantis Radix, Lycii Fructus, Cervi parvum cornu, Ginseng Radix Rubra, and Cassiae cortkex extracts.

An amount ratio of the extracts may be within the above-described range.

Hereinafter, the present invention will be described in further detail with is reference to the following examples.

### Preparation Example

Corni Fructus, Angelica gigantis Radix, Lycii Fructus, Cervi parvum cornu, Ginseng Radix Rubra, and Cassiae cortkex were dried and extracted by adding, to each ingredient, 30% ethanol having a weight 10 times that of each ingredient for 3 hours or more. Each extract was filtered through a filter, concentrated in a vacuum, and lyophilized under reduced pressure, and then 25 parts by weight of a Corni Fructus extract, 25 parts by weight of an Angelica gigantis Radix extract, 25 parts by weight of a Lycii Fructus extract, 10 parts by weight of a Cervi parvum cornu extract, 10 parts by weight of a Ginseng Radix Rubra extract, and 5 parts by weight of a Cassiae cortkex extract were mixed together, thereby completing the preparation of an extract composite.

### Experimental Example

1. Establishment of Benign Prostatic Hyperplasia Model Accompanied by Detrusor Overactivity (Transabdominal Route)

Male Sprague-Dawley rats weighing 250 g to 300 g were used as experimental animals. The experimental animals were anesthetized with intramuscular injection of ketamine (40 mg/kg) and xylazine (20 mg/kg), and then the lower abdomen was vertically incised in a supine position to detach the bladder neck and the periurethra. A control was sutured without ligation of the urethra after the bladder neck and the periurethra were detached. For an experimental group, a polyethylene catheter was placed in the same direction as that in which the urethra was positioned, and then the bladder neck was appropriately tied with the polyethylene catheter and the urethra by using a 3-0 suture. Subsequently, the polyethylene catheter was removed, and the bladder and the urethra were placed at correct positions, followed by suturing of the abdominal incision site. Intervals of day and night were set to 12 hours and all the rats were freely fed food and water according to the prescribed protocol.

2. Urodynamic Examination

The experimental animals were anesthetized with urethane (12 mg/kg), and then the urethral orifice was identified in a supine position. A polyethylene catheter was placed in the bladder through the urethra and then the bladder was emptied and fixed. The catheter was connected to a perfusion pump and a pressure transducer, followed by stabilization for about 10 minutes, and then a bladder internal pressure was set to 0 mmHg. The bladder internal pressure was measured while injecting a physiological saline solution into the bladder at a rate of 10 ml/hour. During observation of micturition pressure, perfusion was stopped when detrusor contraction started or a physiological saline solution was leaked around the urethral orifice. Maximum micturition pressure, the number of detrusor contractions, and intervals therebetween were measured in a minimum of three repetitive micturition cycles

3. Histologic Examination

Bladder tissue samples were fixed with 4% paraformaldehyde at 4° C. for 1 day, and inserted into paraffin. For Masson's trichrome staining to observe the bladder muscle, 7 μm-thick slice sections were prepared.

Details for each group are as follows and all animal experiments were carried out in accordance with the research ethics guidelines of the Animal Experimentation Ethics Committee of the present medical center.

1) Normal: Male Sprague-Dawley rats, in which detrusor overactivity had not been induced, were not administered any drug and not treated

2) OAB: A mouse model with benign prostatic hyperplasia accompanied by detrusor overactivity was not adminstered a drug and not treated

3) A preparation example (400 mg/kg)-administered group: The extract of Preparation Example was diluted in 1 cc of distilled water and the resulting extract was administered via oral gavage twice a day for 4 weeks from week 2 after partial occlusion.

Experimental Results

1) Comparison Between Maximum Micturition Pressure Periods by Urodynamic Examination

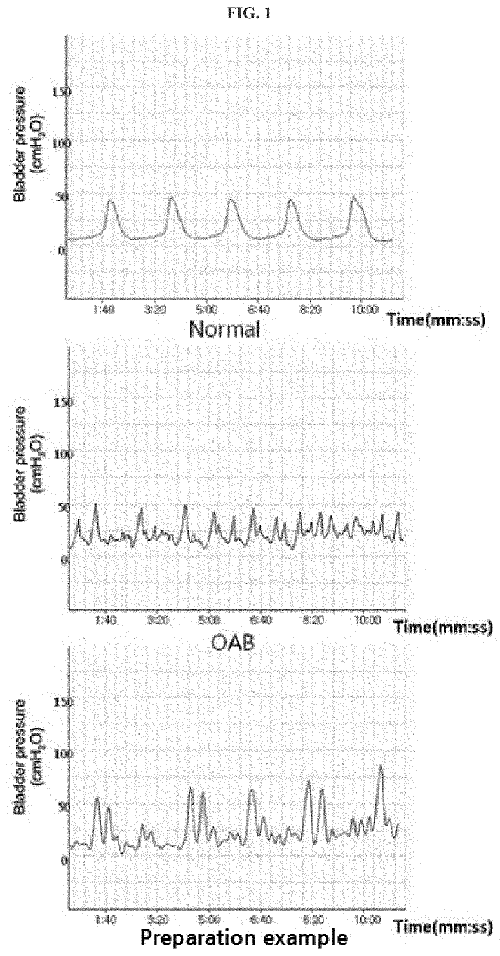

Referring to FIG. 1, it can be confirmed that, when compared to the OAB group, the maximum micturition pressure period is more regular and a difference between maximum and minimum micturition pressures is greater in the preparation example-administered group.

2) Extraction of Bladder after the Examination and Histologic Examination; Observation of Histologic Characteristics of Bladder Mucosa, Submucosa, and Detrusor

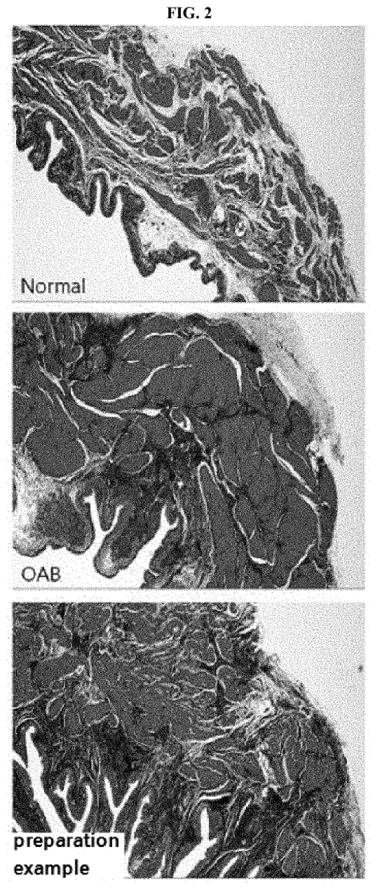

Referring to FIG. 2, it can be confirmed that bladder smooth muscle contraction is significantly decreased in the preparation example-administered group, as compared to the OAB group.

3) Comparison in Concentrations of Inflammatory Cytokines Such as TNF-α, II-6, and IL-8 in blood serum by ELISA

After the experiments were completed, blood was collected by cardiocentesis and centrifuged to separate serum. The separated serum was used for measuring secretion amounts of TNF-α, IL-6, and IL-8 by using an ELISA system. To measure secretory cytokines, 50 μl of a biotinylated antibody was put on a plate coated with an antibody against each cytokine to be measured, 50 μl of a cell culture supernatant was put thereon, and then the resulting plate was maintained at room temperature for 2 hours and then washed. Subsequently, 100 μl of a streptavidin-HRP solution was put onto the plate, the plate was maintained at room temperature for 30 minutes, 100 μl of a 3,3′-5,5′ tetramethylbenzidine (TMB) substrate solution was added thereto to allow a reaction to occur therebetween, a stop solution was added thereto, and absorbance was measured at 450 nm to create a standard curve, to calculate the concentration of the corresponding secretory cytokine.

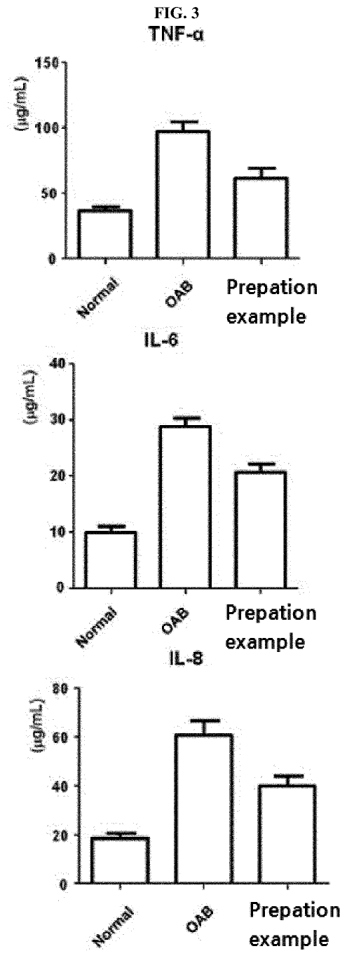

Referring to FIG. 3, it can be confirmed that the concentrations of the inflammatory cytokines are considerably decreased in the preparation example-treated group, as compared to the OAB group.

4) Comparative Observation of Detrusor Contraction-Associated RhoA Protein Expression, RhoA/ROCK mRNA (RhoA/ROCK Pathway) Expression. And NOS

RhoA protein expression or RhoAROCK mRNA (RhoA/ROCK pathway) expression: Rho kinase is activated by GTPase RhoA, and the activated Rho kinase increases the phosphorylation of MLC by inhibition of MLC phosphatase, thus causing detrusor contraction without an increase in intracellular Ca²⁺ concentration. To examine the degree of RhoA protein expression in the bladder in the control and the experimental group. RhoA monoclonal antibodies were used for immunohistochemical staining.

Cryopreserved frozen bladder tissues were excised and attached to slides, and then to avoid a non-specific staining reaction, the tissues were reacted with non-immunized horse serum at room temperature for 30 minutes, and then RhoA monoclonal antibodies diluted 1:200 were added thereto and allowed to react at 4° C. for 24 hours, followed by immunostaining using an avidin-biotin complex kit and observation using an optical microscope.

NOS measurement (western blotting): The collected tissue was sectioned, put in 250 μl of RIPA buffer (25 mM Tris-HCl [pH 7.6], 150 mM NaCl, 1% NP-40, 1% sodium deoxycholate, 0.1% SDS, protease inhibitor cocktail), crushed using a homogenizer, put onto ice, and then maintained at 4° C. for 16 hours. The tissue sections were centrifuged at 13,000 rpm and 4° C. for 15 minutes, and only the supernatant was transferred into a new tube, quantified using a Bradford protein assay, and then stored at −70° C. 8% and 15% SDS-polyacrylamide gels were prepared and electrophoresed using 50 μg of proteins at 100 V for 1.5 hours. After the electrophoresis, the gels were transferred to PVDF membranes at 70 V for 2 hours, and blocked with a 5% skim milk solution for 1 hour. The resulting gels were allowed to react with eNOS, nNOS, and beta-actin at 4° C. for 16 hours, washed with a TBST solution three times each for 10 minutes, allowed to react with anti-mouse IgG-HRP (1:2,000, 1:5,000, Invitrogen) or anti-rabbit IgG-HRP (1:2,000, Invitrogen) at room temperature for 1 hour, washed with a TBST solution three times each for 10 minutes, and then allowed to react with an ECL plus solution for 1 minute, and thicknesses of bands showed by exposing the films to light were compared with each other, thereby identifying the presence or absence of the expression of proteins or a difference therein.

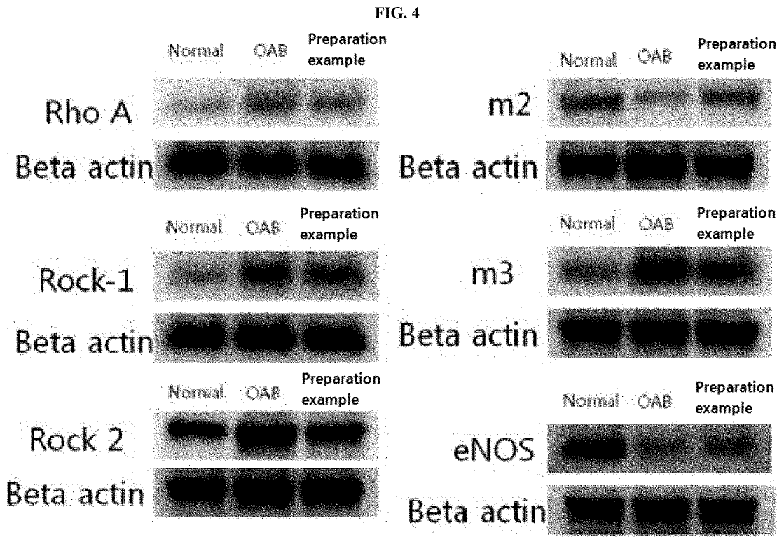

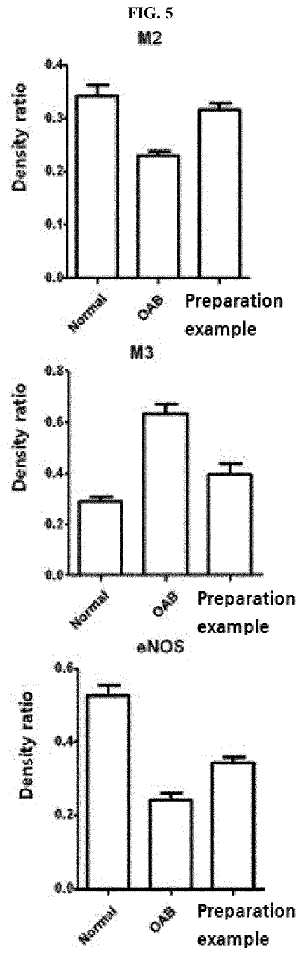

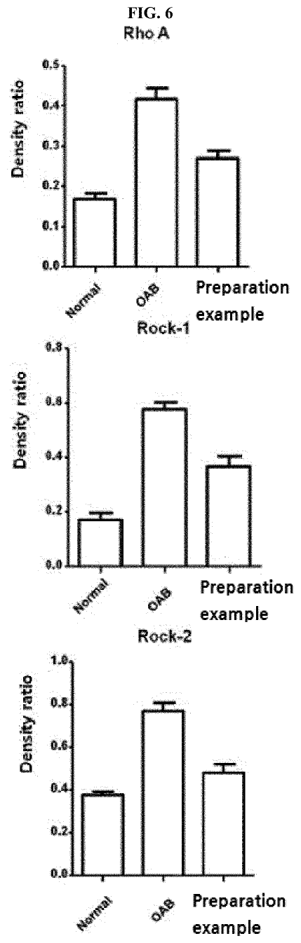

Referring to FIGS. 4 to 6, it can be confirmed that the protein and mRNA expression levels of contraction-associated factors, i.e., m2 and eNOS, are increased, and the protein and mRNA expression levels of contraction-associated factors m3, RhoA, Rock-1, and Rock-2 are reduced, and thus the preparation example-administered group exhibits reduced contraction compared to the OAB group.

1-9. (canceled)

10: A method for preventing or treating overactive bladder, the method comprising administering a composition to a subject in need thereof, the composition comprising extracts comprised of a Corni Fructus extract, an Angelica gigantis Radix extract, a Lycii Fructus extract, a Cervi parvum cornu extract, a Ginseng Radix Rubra extract, and a Cassiae cortkex extract.

11: The method of claim 10, wherein the composition further comprises an alpha-1A adrenoceptor antagonist.

12: The method of claim 11, wherein the overactive bladder is a complication of benign prostatic hyperplasia.

13: The method of claim 12, wherein the overactive bladder is caused by secondary bladder degeneration occurring due to bladder outlet obstruction.

14: The method of claim 12, wherein the subject has the overactive bladder which is not relieved even after bladder outlet obstruction disappears.

15: The method of claim 12, wherein the extracts comprise 20 parts by weight to 30 parts by weight of the Corni Fructus extract, 20 parts by weight to 30 parts by weight of the Angelica gigantis Radix extract, 20 parts by weight to 30 parts by weight of the Lycii Fructus extract, 5 parts by weight to 15 parts by weight of the Cervi parvum cornu extract, 5 parts by weight to 15 parts by weight of the Ginseng Radix Rubra extract, and 2 parts by weight to 8 parts by weight of the Cassiae cortkex extract, based on 100 part by weight of the total extracts.

16: The method of claim 12, wherein the composition is included in a pharmaceutical composition.

17: The method of claim 16, wherein the pharmaceutical composition further comprises at least one of a carrier, a diluents, an excipient.

18: The method of claim 12, wherein the composition is included in a health functional food, and the health functional food is ingestible when the alpha-1A adrenoceptor antagonist is administered.

19: The method of claim 12, wherein the alpha-1A adrenoceptor antagonist is selected from the group consisting of terazosin, doxazosin, tamsulosin, alfuzosin, and a combination thereof.

20: The method of claim 10, wherein the overactive bladder is caused by secondary bladder degeneration occurring due to bladder outlet obstruction.

21: The method of claim 20, wherein the subject has the overactive bladder which is not relieved even after bladder outlet obstruction disappears.

22: The method of claim 20, wherein the extracts comprise 20 parts by weight to 30 parts by weight of the Corni Fructus extract, 20 parts by weight to 30 parts by weight of the Angelica gigantis Radix extract, 20 parts by weight to 30 parts by weight of the Lycii Fructus extract, 5 parts by weight to 15 parts by weight of the Cervi parvum cornu extract, 5 parts by weight to 15 parts by weight of the Ginseng Radix Rubra extract, and 2 parts by weight to 8 parts by weight of the Cassiae cortkex extract, based on 100 part by weight of the total extracts.

23: The method of claim 20, wherein the composition is included in a pharmaceutical composition.

24: The method of claim 23, wherein the pharmaceutical composition further comprises at least one of a carrier, a diluents, an excipient.

25: The method of claim 20, wherein the composition is included in a health functional food, and the health functional food is ingestible when an alpha-1A adrenoceptor antagonist is administered.
